(19) United States
(12) Patent Application Publication
(10) Pub. No.: US 2005/0196321 A1
(43) Pub. Date: Sep. 8, 2005

(54) Fluidic programmable array devices and methods

(76) Inventor: Zhili Huang, Plainsboro, NJ (US)

Correspondence Address: FISH & RICHARDSON PC, 225 FRANKLIN ST, BOSTON, MA, 02110, US

(21) Appl. No.: 10/858,812

(22) Filed: Jun. 3, 2004

Related U.S. Application Data
(60) Provisional application No. 60/549,336, filed on Mar. 3, 2004.

(51) Int. Cl. C12M 1/34; G01N 33/48

(52) U.S. Cl. 422/68.1; 435/287.2

(57) ABSTRACT

This invention relates to array devices and methods for their fabrication. More specifically, the array devices have automatic flow control, probe array configuration, and dynamic chemical or biochemical reaction for rapid chemical or bio-molecule detection.

### CROSS-REFERENCE TO RELATED APPLICATIONS

This application claims priority to U.S. Provisional Patent Applications, Ser. No. 60/474,777 entitled “A Programmable Bio-Microarray,” filed May 31, 2003, and Ser. No. 60/549,336 entitled “Fluidic Adapter for Spot Array,” filed Mar. 3, 2004.

### TECHNICAL FIELD

This invention relates to array devices, the control of fluid flow within those device, and methods for their fabrication.

### BACKGROUND

Microarray technology, providing a quick, cost effective, and parallel analysis, has become a powerful and prominent tool for genomic analysis, molecular diagnosis, and drug development. A microarray is essentially an array of spots on a solid substrate with a surface for molecular probe binding. A DNA microarray is typically composed of DNA “probes” that are bound to a solid substrate such as glass. Each spot in the array lattice is deposited many identical probes that are complementary to the gene sample. During hybridization reaction, “target” DNA samples diffuse passively on the substrate surface, when sequences complementary to the probe will anneal and form a DNA duplex. Hybridized targets can then be read using confocal laser scanning and fluorescence detection. There are two major microarray platforms, one built by synthesis of DNA probes on a substrate in situ and glass slides spotted with complementary DNAs (cDNAs) or oligonucleotides.

The in situ arrays, most commonly GeneChips by Affymetrix of Santa Clara, Calif., are produced by synthesizing oligonucleotides on a glass substrate using photolithographic techniques adapted from the semiconductor industry. Affymetrix produces these preassembled devices for various applications including RNA expression profiling and single nucleotide polymorphism (SNP) detection. Recently, a programmable microarray with light-directed in situ synthesis is introduced. The technology uses a light-modulator matrix, which acts as light valves to control the synthesis of oligonucleotide probes at given positions on the array. This allows any combination of DNA probes to be fabricated on the chip.

The spotted arrays are basically microscope slides that have cDNAs or oligonucleotides deposited on their surfaces. The surfaces are coated with materials such as poly-L-lysine or aminosilane that help attach the DNA molecule probes. The spots are typically less than 200 μm in diameter. Spotted arrays can be produced by robotic equipment in a lab with contact or ink-jet printing methods.

In clinics, high-density microarrays can be used to analyze patient samples and save their lives. But such high density is not important. To determine whether a patient has a particular disease, doctors will need to look only at specific DNA mutations—rarely more than 100. For clinical diagnostic purposes, speed and accuracy are far more important than density.

### SUMMARY

This invention presents fluidic programmable array devices with structures and methods of on-chip flow control, flexible probe configuration, and dynamic chemical or biochemical reaction. In general, in one aspect, the present invention sets forth a fluidic programmable array device having an elastomeric body, a substrate, a recess, a fluidic channel, and two loading wells.

Embodiments of the invention may include one or more of the following features. The array device consists of an elastomeric body and a substrate. The body is an adapter with at least a recess, a fluidic channel, and two loading wells. At the bottom of the device body, the substrate is wedged into and adjacent with the bottom of the device body. The recess is a cylinder with a dome. A spot reservoir comprises the recess and a portion of the substrate at the recess. The substrate basically provides a base of the spot reservoir at the recess for chemical or biological probes to be bound or coupled on it. The substrate may be made of a material, such as silicon, glass, and metal that is able to couple chemicals or molecules on it. The substrate may be coated a material such as ploy-L-lysine, aminosilane, and aldehyde, that is able to couple chemicals or molecules on it. The spot reservoir is connected with the fluidic channel. The fluidic channel can be used for delivering probe and sample solution into the spot reservoir separately. In a preferred embodiment, two fluidic channels are built inside the device body; one is a sample fluidic channel; another is a probe fluidic channel. On both ends of the fluidic channels, loading wells are created. One can be an inlet well, another be an outlet. The dome of the spot reservoir will prevent solution residue at the top edge of the reservoir when the fluidic channel and the spot reservoir are flushed. The body is made of liquid elastomeric material, such as polydimethylsiloxane(PDMS), elastomer or silicone rubber with or without optical transparence.

In a further embodiment of this invention, the array device may consist of more than the one recess to form an array of the spot reservoirs with the substrate and more than the one sample and the one probe fluidic channels to form a fluidic network. The two fluidic channels are intersected at the each spot reservoir. Each spot reservoir is able to be isolated with each other by microvalves pinching the one fluidic channel or all fluidic channels.

In general, in another aspect, the present invention features a structure in a device body made of elastomeric material to realize the fluidic manipulation. The structure includes a recess and a fluidic channel. The recess having an elongated shape with a cylindrical top is created in the device body for an actuator to approach the fluidic channel. A deformable membrane is formed between a gap of the fluidic channel and the cylindrical top of the recess. In this embodiment, the recess is underneath the fluidic channel, but it can approach the fluidic channel from the top or the sides around the fluidic channel. By activation and non-activation of the actuator the deformable membrane can be pinched or return to its initial form. The fluidic channel, the deformable membrane, and the actuator compose an integrated pinch microvalve.

One embodiment of a structure to form the pinch microvalve inside the device body comprises the recess, the fluidic channel, the deformable membrane, and a linear motion actuator. The linear motion actuator with a cylindrical tip is inserted into the recess. When the linear motion actuator moves forward or backward, the deformable membrane is pinched or restored, so the fluidic channel is closed or re-opened. Therefore a pinch microvalve is integrated inside the device.

Varies linear actuators can be used to realize the pinch microvalve. Embodiments of the linear motion actuator are a piezoelectric motion component, a magnetostrictive component, a shape memory alloy, and thermopneumatic motion actuator. The piezoelectric or magnetostrictive motion component can be embedded in a cavity formed by the recess and the substrate. By applying an electric field or a magnetic field, the component exhibit a displacement that pinches the fluidic channel. The thermopneumatic motion actuator can be realized in a cavity filled with a liquid and a build-in microheater. The cavity is formed by the recess and a substrate. The thermopneumatic motion actuator integrated in the device body performs an expansion or contraction at the deformable membrane when a current is applied to the microheater or not. The deformation of the membrane will close or re-open the fluidic channel.

At one end of the fluidic channel before the loading well, three pinch microvalves align in series along the fluidic channel. When the actuators move forward and backward in a sequence, the fluidic channel is squeezed or re-opened in the sequence, which propels liquid in the loading well moving forward. The pinch microvalves become a three-stage peristaltic pump.

In general, in another aspect, the invention presents a fluidic programmable array device with an elastomeric body, a spot reservoir, a fluidic channel, and two loading wells for fully automatic flow control, probe configuration, and dynamic chemical or biochemical reaction.

Embodiments of the invention may include one or more of the following features. The array device consists of an elastomeric body with a spot reservoir inside. The body is an adapter with at least a recess, a fluidic channel, and two loading wells. From the bottom of the device body, a pillar is inlaid into the recess. The top of the pillar with the recess in the body forms a cavity for the spot reservoir. The top of the pillar basically provides a base of the spot reservoir for chemical or biological probes to be coupled on it. A through channel is in the center of the pillar to form a fluidic connecter to delivering a solution from outside of the device into the spot reservoir. The spot reservoir is connected with the fluidic channel. On both ends of the fluidic channel the loading wells are created. One is used as inlet, another as outlet. The body is made of liquid elastomeric material, such as elastomer, PDMS, or silicone rubber with or without optical transparence.

Another preferred embodiment of this invention is that the spot reservoir comprises a cavity inside the elastomeric body. A channel in the elastomeric body connects to the cavity from outside of the device. A further embodiment is that a substrate is embedded in the cavity of the spot reservoir. On the substrate there is a center hole coincident with the through channel in the elastomeric body for liquid delivering from outside of the device to the spot reservoir. The top surface of the substrate is capable of coupling chemicals or molecules on it.

In a further embodiment of this invention, the array device may consists of more than the one spot reservoir to form an array of the spot reservoirs and more than the one fluidic channel to form a fluidic network. The spot reservoir along the fluidic channel is connected by the fluidic channel and able to be isolated with each other by the actuator pinching the fluidic channel.

For spot configuration, the spot reservoir is connected with the probe fluidic channel while the sample fluidic channel is blocked from the spot reservoir by the microvalves along the sample fluidic channel. The probe solution is loaded into the inlet well and propelled through the fluidic channel into the spot reservoir by the micropump at the inlet well. The molecules or chemicals in the probe solution will be bound or coupled onto the surface of the substrate. This reservoir is configured as a sensor spot to detect the corresponding chemical or biological samples. After the spot reservoir is configured, a wash bath solution can be loaded and pumped through the probe channel to wash away the residual probe solution from the spot reservoir to the outlet well.

For chemical and biological reaction on chip, the probe fluidic channel is disconnected to the spot reservoir by the microvalves along the probe fluidic channel and the sample fluidic channel is connected to it. The sample solution will be pumped to the spot reservoir through the sample fluidic channel by the micropump at the inlet well. A chemical or biological reaction will be occurred between the probe's chemicals or molecules and the sample's chemicals or molecules. After that the wash bath solution will be loaded and pumped through the sample channel to flush away the residual sample solution to the outlet well. With laser induced fluorescence or other detection a positive or a negative reaction between the probe and the sample can be detected.

In general, in another aspect, the invention features a method that includes assembling a mold body and a set of mold components to form a device mold for a device body, casting the device body from the device mold, removing the set of mold components from the device mold and the device body, releasing the device body from the mold body, and assembling the device with the device body and functional components.

Embodiments of the invention may include one or more of the following features. Casting the device may include pouring or injecting a liquid elastomeric material into the device mold. The mold component may have a shape that is complementary of a structure of the device after the mold component is removed from the device. The mold component may include an elongated mold component having a same dimension and shape as a channel in the device after the mold component is removed from the device.

The set of mold components may include a first mold component and a second elongated mold component, the first mold component having a shape configured so that a recess is formed in the device when the first mold component is removed from the device, the second elongated mold component having a same dimension and shape as a channel in the device after the second mold component is removed from the device. The first and the second mold component may be spaced at a distance when the device mold is assembled so that a deformable membrane is formed in the device body between the recess and the channel when the first and the second mold component are removed from the device body.

The set of mold components may include an elongated mold component and a mold component with a dome head. On the dome head there is a slot with a dimension substantially the same as a dimension of the elongated mold component. The mold component with its dome supports the elongated mold component at a predetermined position relative to the mold body when the device mold is assembled. Assembling the device mold may include inserting the elongated mold component into the slot.

The set of mold components may include a castable mold component having a shape suitable for forming a cavity and an elongated mold component suitable for forming a channel connecting the cavity in the device. On the castable mold component there is a slot with a same dimension and shape as the elongated mold component. The mold body may have a sidewall with a slot at a predefined position relative to the mold body. Assembling the device mold may include inserting the elongated mold component through the slot on the castable mold component and the slot on the sidewall of the device mold. The elongated mold component supports and aligns the castable mold component in the device mold. To embed a substrate inside the cavity, the substrate may be stuck on the bottom of the castable mold component. After the device body is released from the device mold, a physical condition such as temperature may apply to the device body. The castable mold component inside the device body will be melted from a solid phase to a liquid or a gaseous phase, so that it can be removed from the device body. Therefore the cavity is built inside the device body and the substrate is embedded.

An embodiment for the device assembly, functional components, such as pillars, a slide substrate, and plugs will be put into the device body. The pillar and the substrate will supply a solid surface for chemicals or molecules coupling. The plugs will close part of the channels that left open when the elongated mold components were remove from the device body. Thus, the functional device is built with fully automatic flow control, probe configuration, and dynamic chemical or biochemical reaction.

An advantage of the present invention of the fluidic programmable array device is that fully automatic system will avoid any intervention and contamination while performing the assay. Many factors need to be considered for microarraying work in a laboratory, such as temperature, humidity, and particles in air. With the microvalve and the micropump integration, once probe solutions and sample solutions are applied to the device, all procedures, such as coating, binding, coupling, washing, and reaction, are performed inside the spot reservoirs along the fluidic channels at a certain temperature under computer control. Failures from human intervention are decreased to minimum. Environmental contaminations are completely eliminated.

A further advantage of above embodiments of the present invention is flexibility and customization of the array device. With the sophisticated fluidic channels, connecters, and spot reservoirs, the probe immobilization and configuration to the spot reservoirs can be performed easily. For example, cystic fibrosis is one of the most common autosomal-recessive disorders effecting one in 2500 births in the Caucasian population. Although there have been over 1000 mutations identified that cause cystic fibrosis, only 25 have been generally recommended for carrier screening. But for different racial groups the recommended mutations may somewhat be different. With the fluidic programming array device presented in this invention, the mutations detected for different racial groups can be easily configured in the array device.

Another advantage of the above embodiments of the present invention is parallel detection of different samples and controls simultaneously with same or different mutations in same conditions. For example, in gene screening analysis, it is common that mutant-type, wild-type, and negative control samples are detected. With the fluidic programming array device, same kind of probes can be immobilized in the spot reservoirs along the probe fluidic channel; and different samples and controls can be loaded into the different sample channels, and reacted with the probes along the sample channel. The reaction and detection of the different samples and controls can be performed simultaneously at the same condition.

A further advantage of the above embodiments of the present invention is dynamic reaction in channel. Currently, microarray chip have been widely used for DNA analysis and disease diagnosis. Various cDNA or oligonucleotides probes are synthesized or spotted on a solid substrate. Only the complementary probes react with specific DNA sample fragments coordinated with the probes. But the size of the probe spots is about 0.2 mm in diameter and the DNA sample is dropped onto the substrate to cover the all probe area. The concentration of the target DNA fragments is required to be high enough to make the hybridization reaction between the specific DNA fragment and the complementary probe occurred in the area of 0.2 mm in diameter, otherwise the specific DNA fragment may fail to match the complementary probe. This requirement makes that the efficiency of sample usage is very low and the reproducibility of the analysis and diagnosis is poor in current microarray technology. In embodiments of the present fluidic programmable array devices, the spot reservoirs are integrated along the fluidic channel. During hybridization reaction the target DNA sample is propelled along the fluidic channel forward and backward at a certain flow rate by a micropump. Because of sample solution movement, the each specific DNA fragment will find the complementary DNA probes at the spot reservoirs along the channel. With such dynamic reaction, the requirement for large amount and high concentration of sample is eliminated, the detection sensitivity and accuracy is increased. Therefore, the reproducibility and the efficiency will be highly increased.

Other features, objects, and advantages of the invention will be apparent from the description and drawings, and from the claims.

### DESCRIPTION OF DRAWINGS

FIG. 1 is a perspective view of a fluidic programmable array device having spot reservoirs, probe fluidic channels, and sample fluidic channels, in accordance with one embodiment of this invention.

FIG. 2 is a top view of the array device shown in FIG. 1.

FIG. 3 is a sectional view of the array device along a plan 3 in FIG. 2.

FIG. 4 is a detail sectional view of one of spot reservoirs with two pinch microvalves on its both sides in the FIG. 3.

FIG. 5 is a detail cross sectional view of one of the pinch microvalves in the FIG. 4 with a piezoelectric actuator instead in accordance with one embodiment of this invention.

FIG. 6 is a detail cross sectional view of one of the pinch microvalves in the FIG. 4 with a thermopneumatic actuator instead in accordance with one embodiment of this invention.

FIG. 7 is a perspective view of a fluidic programmable array device having spot reservoirs, fluidic channels, and fluidic connecters in accordance with one embodiment of this invention.

FIG. 8 is a top view of the array device shown in FIG. 7.

FIG. 9 is a sectional view of the array device along a plan 9 in FIG. 8.

FIG. 10 is a detail sectional view of one of spot reservoirs in the FIG. 9, where the array device is on a programming stage with a capillary adapter.

FIG. 11 is a detail sectional view of one of spot reservoirs in the FIG. 9, where the array device is on a programmning stage with a small well underneath.

FIG. 12 is a sectional view of one of spot reservoirs in the FIG. 9, where the array device is on a reaction stage.

FIG. 13 is a cross sectional view of one of the spot reservoirs with a solid substrate embedded in the spot reservoir and a fluidic connecter integrated with device body in accordance with one embodiment of this invention.

FIG. 14 shows a perspective view of a device mold used for fabricating the fluidic programmable array device shown in FIG. 7.

FIG. 15 is a top view of the device mold shown in the FIG. 14.

FIG. 16 is a cross sectional view of the device mold shown in the FIG. 14.

### DETAILED DESCRIPTION

### DEFINITIONS

The term “spot reservoir” as used herein refers to a small liquid cavity. The spot reservoir may be connected with a fluidic channel. Dimensions of the spot reservoirs are from millimeters to micrometers.

The term “array device” as used herein refers to a device having an array structure composed of the spot reservoirs.

The term “fluidic programmable array device” as used herein refers to an array device in which spot reservoirs can be configured with different probes through fluidic channels.

The term “linear actuator” as used herein refers to a component that transforms electrical, magnetic, or thermal energy into a controllable linear motion. A linear actuator used in this present invention may be an electric-mechanical linear motion actuator, a piezoelectric component, a magnetostrictive component, a shape memory alloy, or a thermopneumatic motion actuator.

The term “pinch microvalve” as used herein refers to a structure composed of a channel a recess, and a linear actuator. The linear actuator is placed through the recess or integrated close to the channel. A gap between the recess and the channel forms a deformable membrane when the device body is fabricated from elastomeric material. The linear actuator is capable of moving forward or backward, or upward and downward. When the linear actuator is activated, it exhibits a displacement to the deformable membrane that pinches the channel and closes the channel. When the activation is removed from the linear actuator, the deformable membrane will restore to its initial form for elastomeric property of the device body. Then the channel is re-opened.

The term “elastomeric material” as used herein refers to a material that can cure by mixing a liquid base and a curing agent at a certain ratio. After solidification, the elastomeric material forms a structure having features that accurately reproduces features of the device mold and mold components. Other properties of the elastomeric material are good thermal stability, ability to repel water and form watertight seals, and flexibility. All these properties make the elastomeric material an important engine for fluidic and microfabrication applications. Examples of elastomeric materials are polydimethylsiloxane (PDMS), liquid silicone rubber, room temperature vulcanizing (RTV) rubber, polymeric rubber, or elastoplastic.

The term “castable mold component” refers to a mold component made from one or more reversible, soluble, or sublimable materials. Reversible material refers to a material that is in solid phase at a certain temperature, but changes to liquid phase upon changes in the environment condition(s). Examples of the reversible materials are gel, fusible alloy, and eutectic alloy. Soluble material refers to a lipid material that is in solid at room temperature, but is soluble when upon contact with a solvent. Examples of soluble materials are soap, wax, sterols, and triglycerides. Sublimable material refers to a material that is in solid phase at a certain temperature or pressure, but changes to vapor phase upon changes in the environment condition(s), such as when heated or when the environment pressure is reduced. An example of sublimable material is ammonium salt, such as ammonium chloride (NH₄Cl). A castable mold component is molded to a certain shape that is a complementary of a structure to be fabricated inside a device. A castable mold component can be removed from the device body after the device is molded.

The term “elongated mold component” refers to a mold component having an elongated shape, such as a wire, a rod, or a sheet. A sheet may have a high aspect ratio in which the width and length are larger than the thickness. An elongated mold component can be made from steel, plastic, or silicon. An elongated mold component can also be a castable mold component that is cast from a mold having an elongated inner cavity.

This invention presents several array devices and inside structures, which may be used for chemical and molecular analysis. Methods for fabricating the array devices are also presented.

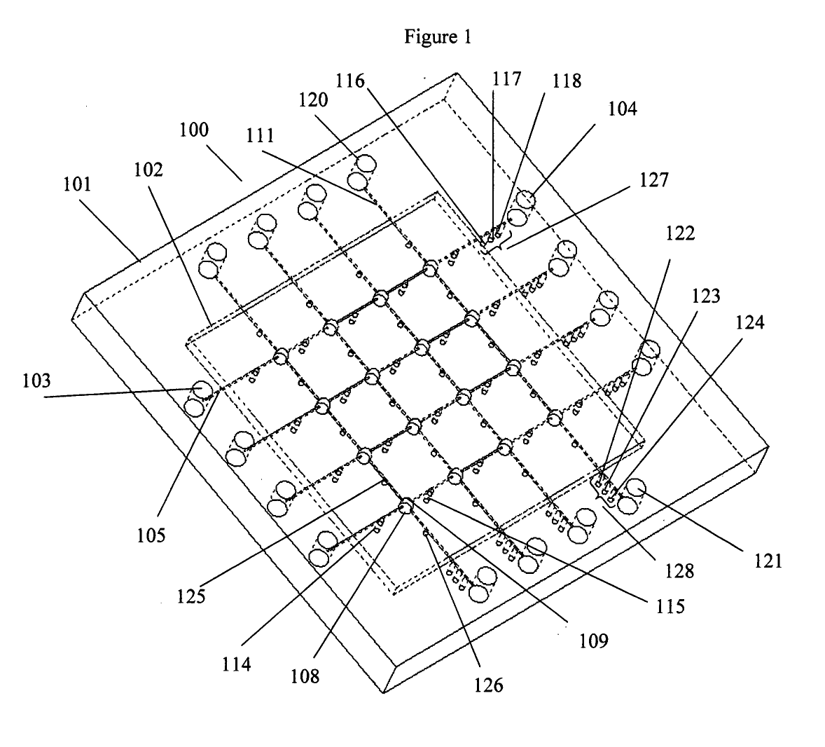

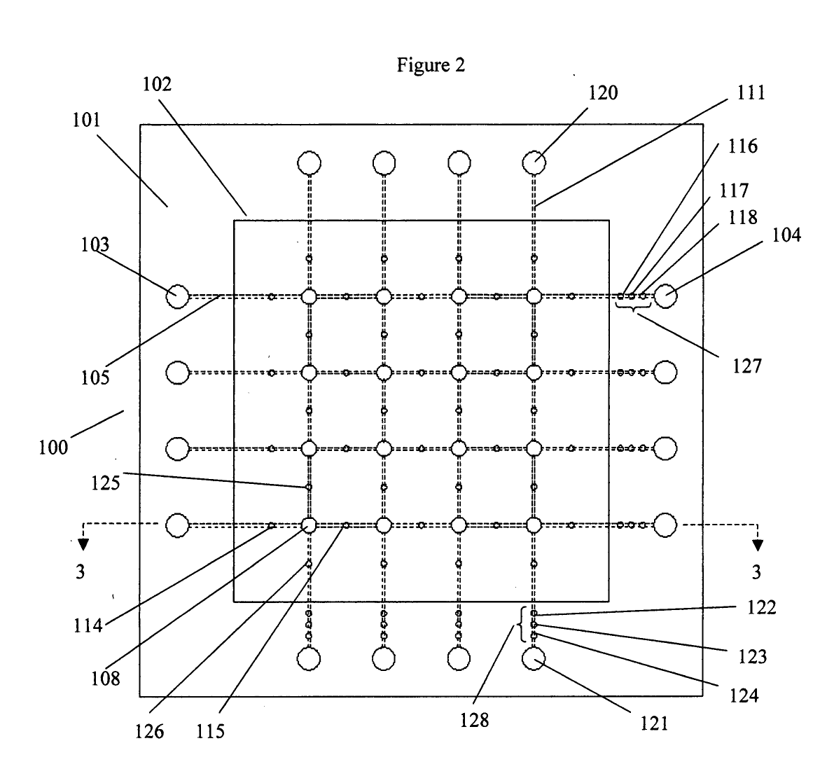

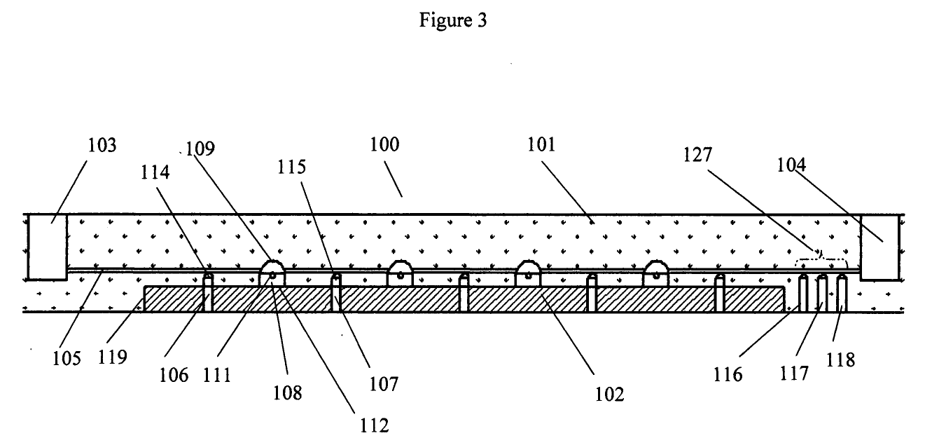

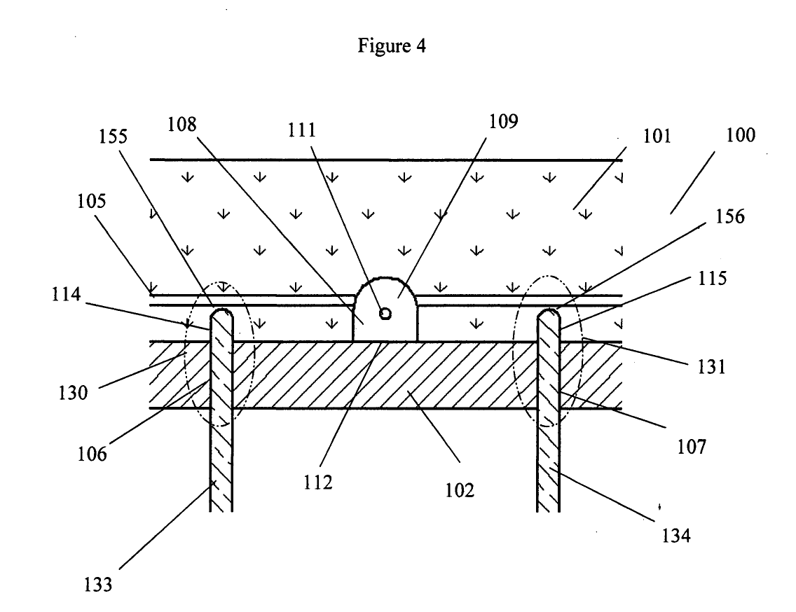

FIG. 1 shows a perspective view of a fluidic programmable array device 100 with 4×4 spot reservoirs 108. FIG. 2 shows a top view of the array device 100. FIG. 3 shows a cross sectional views along a plan 3 in FIG. 2. FIG. 4 shows a detail sectional view of one of spot reservoirs 108 in with two pinch microvalves 130 and 131 along a fluidic channel 105 in the FIG. 3.

The fluidic programmable array device 100 consists of a body 101 and a substrate 102. Inside the body 101, there are four fluidic channels 105 with their loading wells 103 and 104 for four target samples, four fluidic channels 111 with their loading wells 120 and 121 for four probes, 4×4 spot reservoirs 108 with their domes 109, recesses 114 and 115 for linear actuators 133 and 134 on both sides of the each spot reservoir 108 beneath the fluidic channel 105 consisting of two pinch microvalves 130 and 131, recesses 125 and 126 for linear actuators on both sides of the spot reservoir 108 beneath the fluidic channel 111, and two sets of recesses 116, 117, and 118, and 122, 123, and 124 with linear actuators beneath the fluidic channels 105 and 111 consisting of three-stage peristaltic pumps 127 and 128. The spot reservoir 108 comprises a recess 110 and a portion of the substrate 112 at the recess 110. The area 112 at the spot reservoir 108 on the substrate 102 is served as the base of the spot reservoir 108 for chemical or biological probes to be coupled on it. Other area on the substrate 102 is sealed or bonded with the bottom of the body 101. The substrate 102 may be made of a material, such as silicon, glass, polypropylene, polycarbonate, and metal that is able to couple chemicals or molecules on it. The surface of the substrate 102 may be coated a material such as ploy-L-lysine, aminosilane, and aldehyde, that is able to couple chemicals or molecules on it. The body 101 of the array device 100 is made from elastomeric material, such as polydimethylsiloxane (PDMS), polymeric rubber, silicone rubber, or polymeric plastic. On the bottom of the array area of the body 101 there is a recess 119 for the substrate 102. The device 100 is composed of 4 spot reservoirs 108 in a row along the fluidic channel 105 and 4 in a column along the fluidic channel 111 (4×4 array), but the number of the spot reservoirs 108 can be increased. The diameter of the spots reservoirs 108 can be from millimeters to micrometers. The height of the dome 109 may be from millimeters to micrometers. For example, if the spot reservoirs 108 with the pinch microvalves take 3 mm×3 mm space on the 25.4 mm×25.4 mm (1 inch×1 inch) substrate, an 8×8 spot array can be created in the center of the slide substrate. With microfabrication, it can be very easy to realize a spot reservoir with the microvalves in less than 1 mm×1 mm area. On a 25.4 mm×76.4 mm (1×3 inches) microscope slide, a 24×75 spot array device can be created in the center of the slide substrate. With microfabrication technology and a larger slide, more spot reservoirs can be created on the substrate slide. The spot array can be 50×100 to 100×1000. The dome 109 is a preferred shape for the spot reservoir 108; other shapes of the spot reservoirs 108 can be built. The each spot reservoir 108 can be same size or different sizes.

Pinch microvalve in the array device 100:

As shown in FIG. 4, a deformable membrane 155 between the recess 114 and the channel 105 is formed when the device is fabricated from elastomeric material. On the substrate 102 there is a through hole 106. The recess 114 in the body 101 and the through hole 106 on the substrate 102 are aligned. A linear actuator 133 is passed through. The shape of the tip of the linear actuator 133 is same as the cylindrical top of the cavity 114. The tip of the linear actuator 133 is touched the top of the recess 114 when the linear actuator 133 is not activated. The linear actuator 133 is capable of moving forward or backward, or upward and downward along the recess 114 and the through hole 106 by an electromechanical motion component connected at the end of the linear actuator 133. When the linear actuator 133 is activated and moves forward or upward, it will deform the deformable membrane 155 and pinch the channel 105 at the position of the recess 114. When the linear actuator 133 is released and moves backward or downward, the membrane 155 returns to its initial form, and the channel 105 is re-opened. Therefore the linear actuator 133, the deformable membrane 155, the recess 114, the through hole 106, and the channel 105 form a integrated pinch microvalve 130 to control flow in the channel 105 in the device body 101.

On the other side of the spot reservoir 108 along the channel 105, there are a recess 115 and the deformable membrane 156 in the body 101, and a through hole 107 in the substrate 102, which form another pinch microvalve 131 with a linear actuator 134. Along the channel 111, on both side of the spot reservoir 108, there are recesses 125 and 126 that form pinch microvalves with through holes in the substrate 102 and other two linear actuators. So the spot reservoirs 108 in the device 100 can be separated from each other by these pinch microvalves.

Three-stage peristaltic pump on the array device 100:

At one end of the channel 105 before the loading well 104, three recesses 116, 117, and 118 in series associated with linear actuators form three pinch microvalves along the channel 105, as shown in FIG. 3. When the linear actuators move forward and backward in a sequence, such as 001, 011, 110, 100, and 101 where 1 and 0 represent activated and nonactivated states of the linear actuator, the channel 105 will be closed or open in the sequence at the positions of the recesses 116, 117, and 118 respectively. This sequential movement squeezes fluid moving forward in the channel 105. The three pinch microvalves compose of a three-stage peristaltic pump 127 that propels the fluid in the channel 105 forward from the loading well 104 to the loading well 103. With a reverse sequence of the movements of the linear actuators, the fluid in the channel 105 is propelled backward from the loading well 103 to the loading well 104. At one end of the channel 111 before the loading well 121, three recesses 122, 123, and 124 associated with linear actuators form another three-stage peristaltic pump 128 that propels the fluid in the channel 111 forward from the loading well 121 to the loading well 120 or backward from the loading well 120 to the loading well 121.

Preparation of the substrate surface:

For probe immobilization on the substrate 112 at the spot reservoir 108, as an example of DNA analysis application, the surface of the substrate 102 or the whole solid substrate 102 can be coated with either poly-L-lysine or aminosilane to give an amino group surface or covalent attachment to link the probe by sharing electrons between adjacent atoms. The substrate 102 can be treated with a procedure for slide surface coating in conventional microspot technology. After it is treated, the substrate is wedged into the bottom of the device.

The surface 112 at the spot reservoir 108 can be also treated on chip. The untreated substrate 102 is wedged onto the bottom of the device body 101. The substrate 102 and the recesses 110 form the spot reservoir 108. Coating solutions can be loaded into the loading well 104, and propelled along the channel 105 into the spot reservoir 108. Following the protocal for slide surface coating in conventional microspot technology, the surface 112 can be coated.

Probe programming on the array device 100:

To configure the four spot reservoirs 108 along the each channel 105 with different probes, first, the pinch microvalves at the position of the recesses 106 and 107 are activated to close the channel 105 at both sides of the spot reservoir 108. Second, probe solutions are loaded into the each loading inlet well 121. In this embodiment there are four channels 111 with four loading inlet wells 121 and four loading outlet wells 120. Four different probes can be loaded into the four loading inlet wells 121 to configure the four spot reservoirs 108 along the each channel 111 respectively. Third, the three-stage peristaltic pump 128 propels the probe solution through the channel 111 from the loading inlet well 121 to the loading outlet well 120. When the probe solutions flow into the spot reservoirs 108 along the channels 111, the surface of the substrate 112 at the spot reservoirs 108 will absorb or couple the molecules or chemicals of the probes by ionic interaction or covalent attachment. Therefore the spot reservoirs 108 along the channel 111 are configured with one of the four molecular or chemical probes. During the probe configuration the probe solution can be pumped forward and backward along the channel 111 by the three-stage peristaltic pump 128 at a controllable flow rate. This dynamic flow of the probe solution will increase efficiency of the reaction between the surface 112 at the spot reservoir 108 and the probe solution, and the usage efficiency of the probe solution. After the reaction between the surface 112 at the spot reservoir 108 and the probe solution, the probe solution is propelled into the either loading well 121 or 120 and collected. A wash bath solution can be loaded into the loading well 121 and pumped through the channels 111 and the spot reservoirs 108 to wash the residual probe solutions into the loading outlet well 120. Then the solutions in the loading well 120 can be removed from the device 100.

Hybridization reaction in the array device 100:

After the probe configuration in the spot reservoirs 108, the microvalves at the position of the recesses 106 and 107 are deactivated to open the channel 105 at both sides of the spot reservoir 108. At the same time, the microvalves at the position of the recesses 125 and 126 are activated to close the channel 111 at both sides of the spot reservoir 108. Then, the sample solutions are loaded into the loading inlet well 104. In this embodiment there are the four sample channels 105 with the four loading inlet wells 104 and the four loading outlet wells 103. Four different samples can be loaded into the four loading inlet wells 104. The three-stage peristaltic pump 127 propels the sample solutions through the channels 105 from the loading inlet wells 104 to the loading outlet wells 103. In this embodiment, DNA samples are used to be an example of the applications of the array device 100. When the DNA sample solutions flow into the spot reservoirs 108 along the channels 105, hybridization reaction will be happened in the spot reservoirs 108 between the probes coupled on the surface 112 and the sample solutions. During the hybridization reaction, the sample solutions can be pumped forward and backward along the channel 105 at a certain flow velocity by the three-stage peristaltic pump 127. This dynamic flow of the sample solution will increase efficiency of the reaction between the probes on the surface 112 at the spot reservoir 108 and the sample solution. The efficiency of the sample solution usage will be increased also. After the reaction, a wash bath solution can be loaded into the loading inlet wells 104 and pumped through the channels 105 and the spot reservoirs 108 to wash the unbound sample solutions into the loading outlet wells 103. Then the solutions in the wells 103 can be removed from the device 100.

Hybridization reaction is a preferred example for DNA analysis application, other chemical or biochemical reaction can be also applied in this present array device for chemicals, molecules, cells, or tissues detection and analysis. For example, for protein analysis, antibody probes can be configured in the spot reservoirs, and antigen samples can be loaded into the device. Then antibody-antigen interactions will be occurred in the spot reservoirs.

For the body 101 is optical transparent, the result can be read from the top of the array device 100. The array device 100 can be put on a translation stage with laser induced fluorescence detection. When the translation stage moves, the array device 100 is scanned and the reaction result in the each spot reservoir 108 is read. The body 101 is not necessary to be optical transparent. The substrate 102 can be removed from the device body after the reaction, and put on a commercial-available reader for microarray or microspot to detect the reaction result at the positions of the spot reservoirs 108. The methods for the detection can be confocal laser scanning and sensitive CCD picturing.

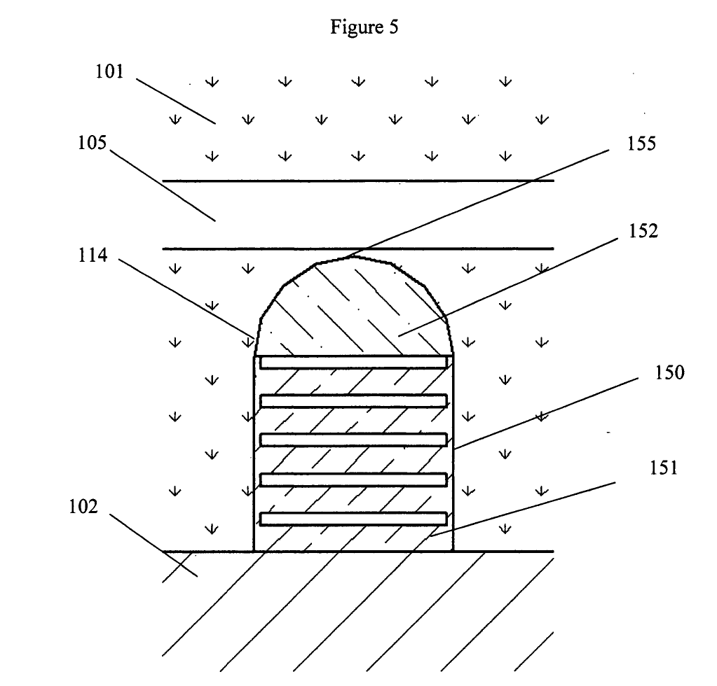

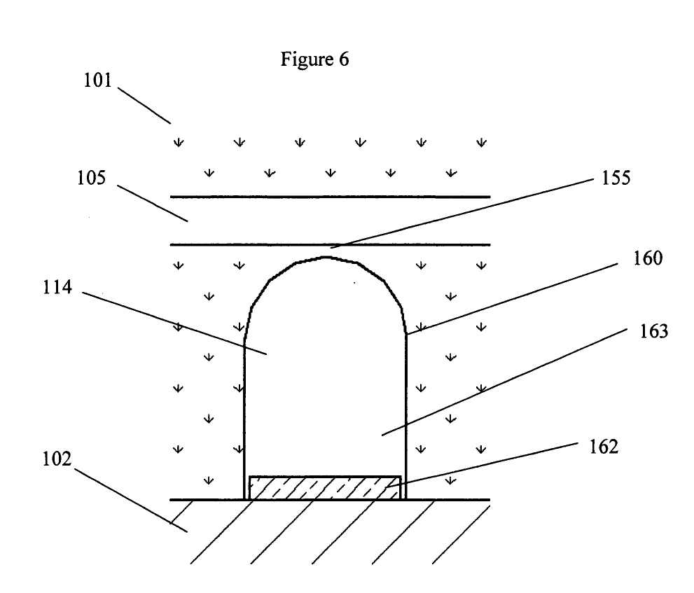

FIGS. 5 and 6 illustrate other two embodiments of integrated pinch microvalves. FIG. 5 shows a pinch microvalve activated by a linear solid microactuator 150. FIG. 6 shows a pinch microvalve activated by a thermopeumatical microactuator 160.

In FIG. 5, as another embodiment of the microvalve by the linear solid microactuator 150, 5 piezoelectric components 151 and a cylindrical pinch head 152 are stacked together to compose the microactuator 150 in the recess 114 on the substrate 102. Piezoelectric movement arises from the dimensional changes generated in certain crystal materials when they are subjected to an electric field. Typical piezoelectric materials are quartz, lead zirconate titanate, lithium niobate, and some polymers, such as polyvinyledene fluoride. The response of piezoelectric materials to changes of the electric field is very quickly and repeatable. When a voltage is applied to the 5 piezoelectric components 151, they generate a linear movement that pushes the cylindrical pinch head 152 upward. The cylindrical pinch head 152 will deform the membrane 155 between the recess 114 and the channel 105. The deformation closes the channel 105. When the voltage is released from the piezoelectric components 151, the membrane 155 will restore to its initial shape by elastomeric property of the device body 101. The channel 105 is re-opened.

Other components instead of piezoelectric components 151 can be also used as the linear solid microactuator 150. Examples of such components are shape memory alloy, electroactive polymer (EAP), and magnetostrictive component.

In FIG. 6 shows another embodiment of the microvalve by the thermopeumatical microactuator 160. The microactuator 160 is composed of a sealed cavity 163 and a microheater 162. The cavity 163 is formed by the recess 114 and the substrate 102, and filled with a low boiling point liquid, such as methyl chloride, or fluorinert. The microheater 162 is built inside the cavity 163. When the microheater 163 is connected to a current, the temperature in the cavity 163 increases, the pressure inside grows because of the gas generating from the liquid-gas phase transitions, and the deformable membrane 155 is inflated. The deformation of the membrane 155 will close the channel 105. The microheater 163 can be a small diode, a resistor, or other integrated semiconductors on the substrate 102. When the current is disconnected to the microheater 163, the temperature drops, and the pressure inside decreases, the membrane will restore to its initial form by elastomeric property of the material of the device body 101. The channel 105 is re-opened.

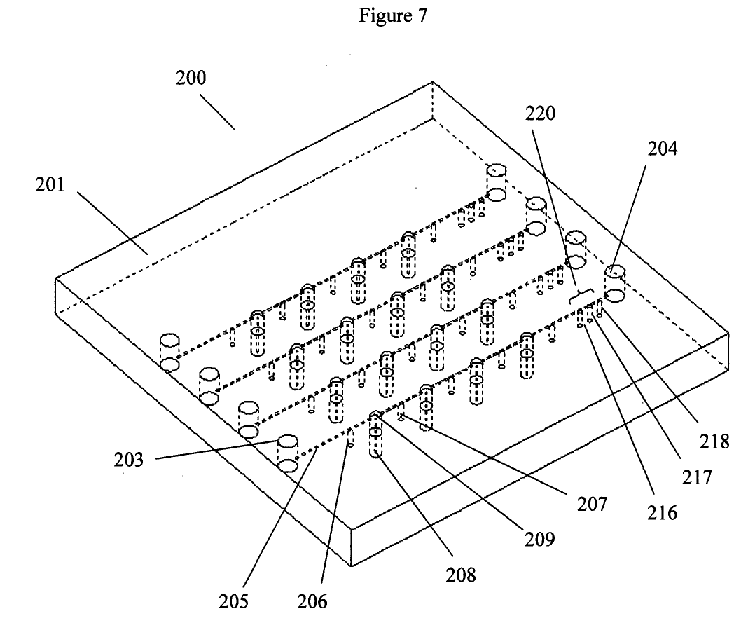

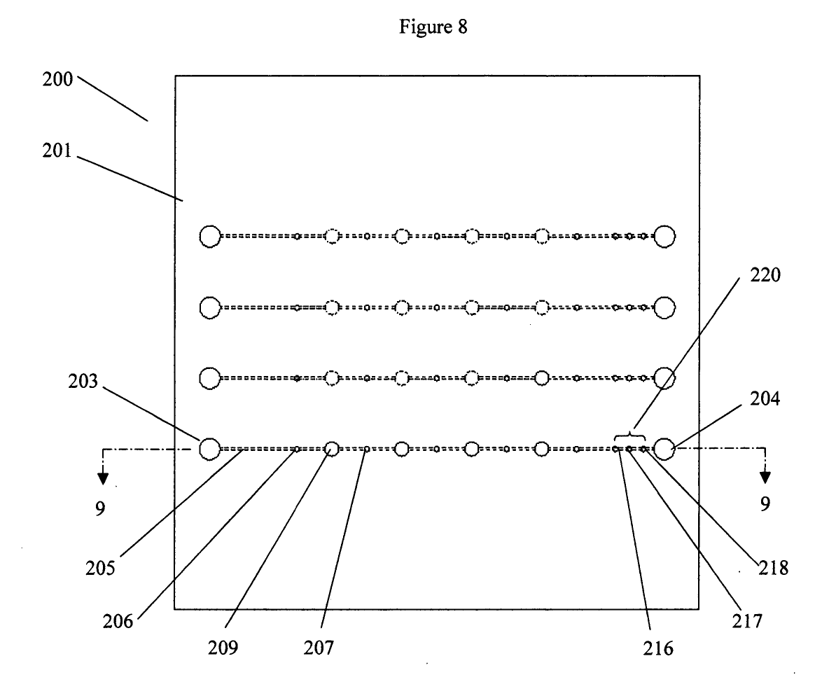

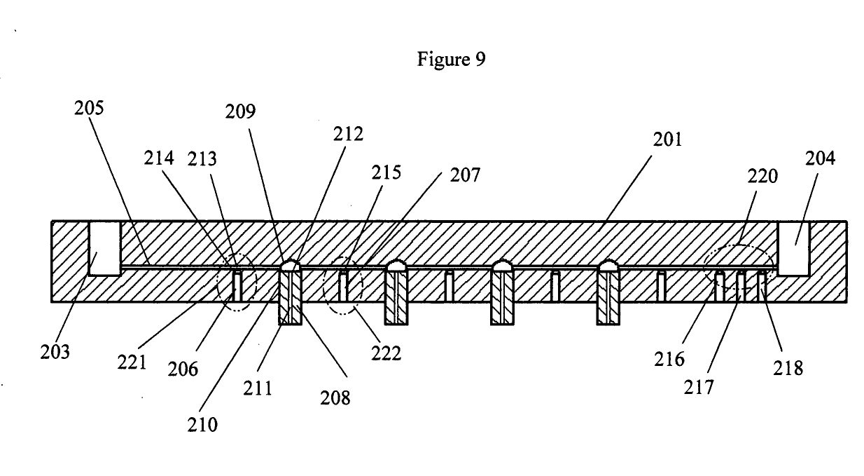

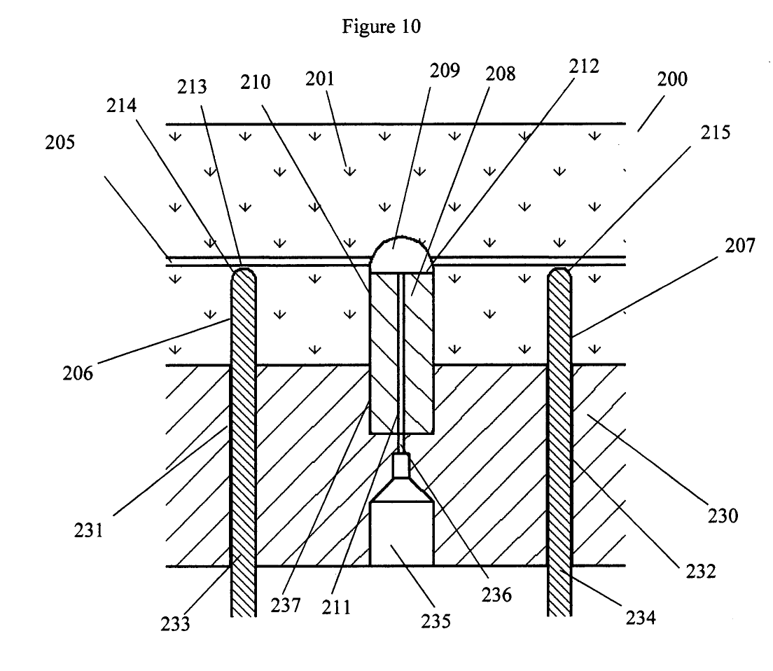

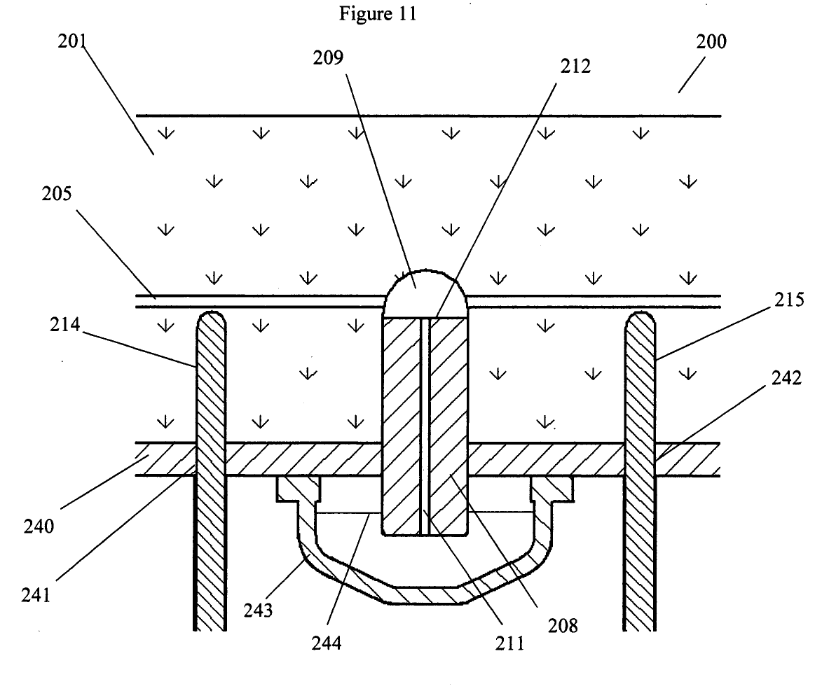

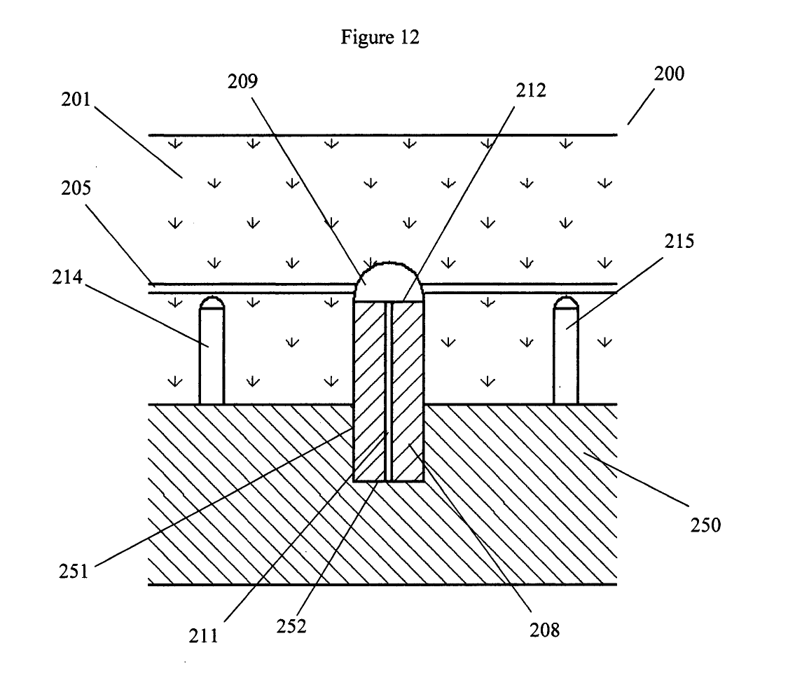

As another embodiment, FIG. 7 shows a perspective view of a fluidic programmable array device 200 with 4×4 spot reservoirs that can be configured with different probes individually. FIG. 8 is a top view of the array device shown in FIG. 7. FIG. 9 shows a cross sectional views along a plan 9 in FIG. 8. FIG. 10 shows a detail of one of spot reservoirs 209 in the sectional view in the FIG. 9, where the array device 200 is on a programming stage 230 with a capillary adapter 235. FIG. 11 shows a sectional view of one of spot reservoirs 209 in the FIG. 9, where the array device 200 is on a programming stage 240 with a probe programming well 243 underneath. FIG. 12 shows a sectional view of one of spot reservoirs 209 in the FIG. 9, where the array device 200 is on a reaction stage 250.

The fluidic programmable array device 200 consists of a body 201 and pillars 208. Inside the body 201, there are four fluidic channels 205 with their loading wells 203 and 204 for four target sample solution, 4×4 spot reservoirs 209, recesses 206 and 207 for linear actuators on both sides of the each spot reservoir 209 along the fluidic channels 205 consisting of pinch microvalves 221 and 222, and a set of recesses 216, 217, and 218 for linear actuators beneath the fluidic channel 205 consisting of a three-stage peristaltic pump 220. The body 201 of the array device 200 is made from elastomeric material, such as polydimethylsiloxane (PDMS), polymeric rubber, or polymeric plastic. The pillar 208 is made from a solid material, such as silicon, glass, polypropylene, polycarbonate, or metal.

On the bottom of the array area of the body 201 there is an array of recesses 210 for accepting the pillars 208. Each pillar 208 corresponds to the each spot reservoir 209 in the body 201. The diameter of the recess 210 equals to the diameter of the pillar 208. When the array device 200 is assembled, the pillar 208 is wedged into the recess 210. The contact surface between them forms a watertight sealing. The top dome of the recess 210 and the top surface 212 of the pillar 208 form the spot reservoir 209. The top surface 212 of the pillar 208 will serve as the base of the each spot reservoir 209. In this preferred example, the pillar 208 and the recess 210 are in cylindrical shape, but other shape can be also applied.

The 4×4 spot array device 200 shown in FIG. 5 is a preferred example, but the one spot reservoir 209 and the one channel 205 can compose the device 200. Also the number of the spot reservoirs 209 in the device 200 can be increased. The diameter of the pillar 208 can be from millimeters to micrometers. The height of the spot reservoir 209 may be from millimeters to micrometers. For example, if the pillar 208 with the microvalve 221 takes 3 mm along the channel 205, the channel-to-channel space is 4 mm, an 8×6 spot array can be created in a 25.4 mm×25.4 mm (1 inch×1 inch) center area of the device 200. With microfabrication, it can be very easy to realize a pillar and a pinch microvalve in several hundreds micrometers in diameters. If the spot reservoir 209 and the microvalve 221 take a 1 mm along the channel 205 and the space between the channels 205 is 1 mm, in a 25.4 mm×76.4 mm (1×3 inches) center area of the device 200, a 24×75 spot array device can be created. The number of the spot reservoirs 209 in the device 200 is 1800 spots. With microfabrication technology, more spot reservoirs can be created in the device 200. The spot array can be 50×100 to 100×1000. The dome of the spot reservoir 209 is a preferred shape; other shapes for the spot reservoirs 209 can be built. The diameter of the pillar 208 can be from millimeters to micrometers. The each pillar 208 can be same size or different sizes.

The four spot reservoirs 209 in the FIG. 9 are connected by the channel 205. The each spot reservoir 209 can be separated by the pinch microvalves 221 and 222 along the channel 205. On each end of the channel 205 there are loading wells 203 and 204 for sample loading. Along the channel 205 there is a micropump 220 to propel the sample solution through the channel 205.

In the preferred embodiment of this invention, the channel 205 is used for sample solution and the channel 211 for probe solution. In practice, the channel 205 can also be assigned for probe solution and the channel 211 for sample.

The surface coating on the surface 212 of the each pillar 208:

The top surface 212 of the pillar 208 serves as the base of the spot reservoir 209. On the center of the each pillar 208, there is a small channel 211. The channel 211 is used to delivery the probe solution into the spot reservoir 209. As an example of DNA analysis application, the surface should be coated with poly-L-lysine, aminosilane or other conventional microarray protocol to allow for efficient probe coupling. The surface treatment can be achieved on each pillar 208 before it is assembled to the device body 201. The procedure can follow the method of the slide surface coating in conventional microspot protocol. Instead, the pillars 208 can be put in a tube soaked with coating solution.

The surface treatment can be also achieved on each pillar 208 after it is assembled to the device body 201. Coating solution can be loaded into the spot reservoir through the channel 211 or the channel 205. The procedure then can follow the method for slide surface coating in conventional microspot protocol.

Pinch valve on the array device 200:

As shown in FIG. 10, between the recess 206 and the channel 205 a deformable membrane 213 is formed when the device is fabricated from elastomeric material. On the socket stage 230 for probe programming there is a through hole 231. The recess 206 in the body 201 and the through hole 231 are aligned. The tip shape of a linear actuator 233 is same as the cylindrical top 214 of the recess 206. The linear actuator 233 is inserted into the recess 206. The tip of the linear actuator 233 is touched the top 214 of the recess 206. The linear actuator 233 is capable of moving forward or backward, or up and down along the recess 206 and the through hole 231 by a linear motion actuator connected at another end of the linear actuator 233. When the linear actuator 233 moves forward or upward, it will pinch the channel 205 at the position of the recess 206. The membrane 213 is deformed and the channel 205 is closed. When the pin actuator 233 is moved backward, the membrane 213 restores to its initial form, and the channel 205 is re-opened. Therefore the linear actuator 233, the membrane 213, the recess 206, the through hole 231, and the channel 205 compose of the pinch microvalve 221 to control flow in the channel 205.

On the other side of the spot reservoir 209 along the channel 205, the pinch microvalve 222 is comprised along the channel 205. Therefore, the spot reservoirs 209 can be separated from each other by these pinch microvalves 221 and 222 along the channels 205.

Integrated pinch microvalves, such as the pinch microvalves built in the device 100 with the piezoelectric components or a thermopneumatic actuator, can be also created in the device 200.

Probe programming on the array device 200:

The each spot reservoir 209 can be configured with different probes. FIG. 10 shows the sectional view of one of the spot reservoir 209 of the array device 200 on the socket stage 230 for probe programming. A socket 237 on the stage 230 is used to accept the pillar 208 on the device 200. The connection between the socket 237 and the pillar 208 is watertight. There is a center hole 236 on the bottom of the socket 237. The diameter of the hole 236 is same as the diameter of the channel 211. The hole 236 is aligned with the center hole 211 on the pillar 208. On the bottom of the socket stage 230, a capillary adapter 235 is built corresponding to the each socket 237. The capillary adapter 235 is used to connect a capillary that deliver a probe solution into the device 200. To configure the spot reservoir 209, the probe solution will be propelled through the capillary, the hole 236, and the channel 211, into the reservoir 209. Then the microvalves 221 and 222 at the position of the recesses 206 and 207 are activated to close the channel 205 at both sides of the spot reservoir 209 to prevent a cross contamination of the probe solution along the spot reservoirs 209 through the channel 205.

When the probe solutions are in the spot reservoirs 209, the surface 212 of the pillar 208 will absorb or couple the molecules or chemicals of the probes by ionic interaction or covalent attachment. Therefore the each spot reservoir 209 is configured with the molecular or chemical probe. After the probe solution is coupled onto the surface 212, the residual probe solution can be withdrawn through the channel 211 and the hole 236 from the spot reservoir 209. The, pinch microvalves 221 and 222 at the position of the recesses 206 and 207 are deactivated to open the channels 205. A wash bath solution can be loaded into the loading wells 204 and pumped through the channels 205 and the spot reservoirs 209 to wash the residual probe solutions into the wells 203. Then the solutions in the wells 203 can be removed from the device 200.

Another embodiment of this invention for probe programming on the array device 200 is shown in FIG. 11 by capillary action. Capillary action is the ability of a capillary to draw a liquid upwards against the force of gravity. It occurs when the lower end of the capillary is placed in a liquid such as water, surface tension pulls the liquid column up. The height h in metres of a liquid column is given by:

$$h = \frac{2 T cos θ}{ρ g r}$$

where T=interfacial surface tension (N/m), θ=contact angle, ρ=density of liquid (kg/m³), g=acceleration due to gravity (m/s²). For a water-filled glass tube in air at sea level, T=0.0728 N/m at 20° C., θ=20°, ρ=1000 kg/m³, g=9.80665 m/s², so the height of the liquid column is given by

$$h \approx \frac{1.4 \times {10}^{- 5}}{r}$$

In this embodiment, the diament of the channel 211 is 0.125 mm, the height of the liquid column will be 112 mm. In the FIG. 11, the pillar 208 is inserted into the probe solution in a small well 244 beneath a socket stage 240 for probe programming. The liquid level in the well 243 is at 244. The height of the pillar 208 is 10 mm. The capillary action is easy to pull the probe solution into the spot reservoir 209 from the liquid level 244 to the surface 212. After the probe solution is in the spot reservoir 209, the microvalves 221 and 222 at the position of the recesses 206 and 207 in the device body 201 and the through holes 241 and 242 on the stage 240 are activated to close the channel 205 at both sides of the spot reservoir 209 to prevent cross contamination of the probe solutions.

Hybridization reaction in the array device 200:

After the probe programming in the spot reservoirs 209, the device 200 can be removed from the programming stage 230 or 240, and put on a reaction stage 250, as shown in FIG. 12 for one of the spot reservoir 209 of the device 200. On the reaction stage 250, there is an array of dead-end socket 251 corresponding to the array of the pillars 208 on the device 200. The end 252 of the socket 251 will seal the end of the channel 211 of the pillar 208. A heater may be attached to the reaction stage 250 to maintain a certain temperature applied to the device 200. The sample solutions are loaded into the inlet wells 204. In this embodiment there are the four channels 205 with the four inlet wells 204 and the four outlet wells 203. Four different samples can be loaded into the four inlet wells 204. The three-stage peristaltic micropump 220 propels the sample solutions through the channels 205 from the wells 204 to the wells 203. In this embodiment, DNA samples are used to be an example of the applications of the array device 200. When the DNA sample solution flows into the spot reservoirs 209 along the channels 205, hybridization reaction will be happened in the spot reservoirs 209 between the probes coupled on the surface 212 and the sample solutions. During the hybridization reaction, the sample solutions can be pumped forward and backward along the channel 205 by the three-stage peristaltic micropump 220. This dynamic flow of the sample solution will increase efficiency of the reaction between the probes on the surface 212 at the spot reservoir 209 and the sample solution. The usage efficiency of the sample solution will also be increased. After the reaction a wash bath solution can be loaded into the wells 204 and pumped through the channels 205 and the spot reservoirs 209 to wash the residual sample solutions into the wells 203. Then the solutions in the wells 203 can be removed from the device 200.

For the body 201 is optical transparent, the result can be read from the top of the array device 200. The array device 200 can be put on a translation stage with laser induced fluorescence detection. When the translation stage moves, the array device 200 is scanned and the reaction result in the each spot reservoir 209 is read. The methods for the detection can be laser induced fluorescence detection, UV absorption detection, or other measurements.

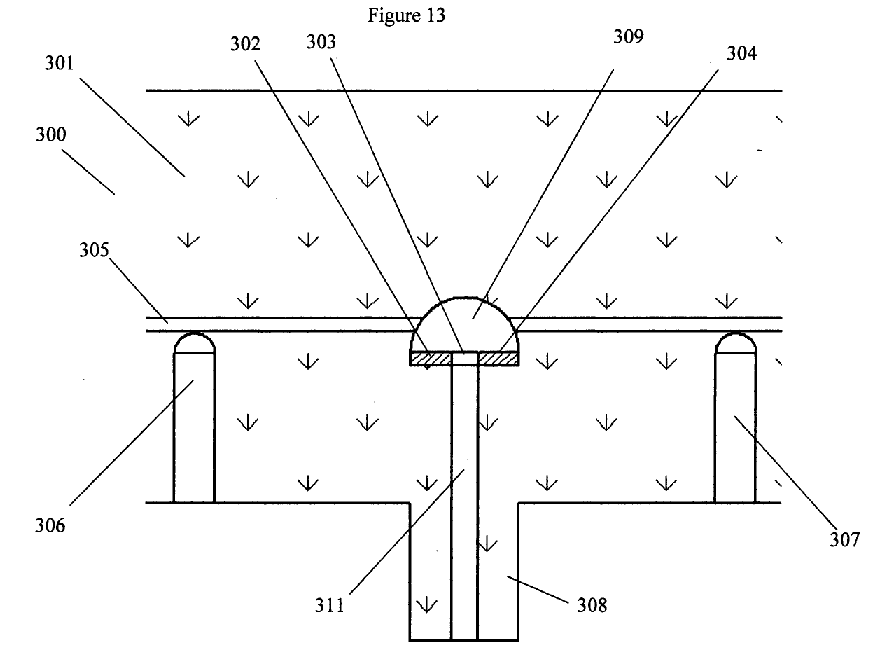

FIG. 13 shows a spot reservoir 309 in an array device 300. The device 300 is similar to the device 200 except that the spot reservoir 309 is embedded in a device body 301, the pillar 208 is replaced by an embedded substrate 302 in the spot reservoir 309, and the channel 211 in the pillar 208 is integrated as a channel 311 with a fluidic connector 308 inside the device body 301. The device body 301 of the device 300 is made from elastomeric material, such as polydimethylsiloxane (PDMS), polymeric rubber, or polymeric plastic. The substrate 302 is made from a solid material, such as silicon, glass, polypropylene, polycarbonate, or metal.

In the device 300, recesses 306 and 307 and a channel 305 associated with linear actuators compose of pinch microvalves same as the microvalves 221 and 222 in the device 200. The channel 305 connects the spot reservoirs 309. Numbers of the channel 205 and the spot reservoirs 309 can be designed in the device 300 according to application requirements.

On the substrate 302, there is a small hole 303 connected with the channel 311. The channel 311 is used to delivery the probe solution into the spot reservoir 309. For probe immobilization on the surface 304 in the spot reservoirs 309, the surface 304 could be either amine- or lysine-coated to absorb the probe by ionic interaction or covalent attachment to link the probe by sharing electrons between adjacent atoms.

Other procedures and operations of the device 300 are similar to those of the device 200.

Methods for device fabrication:

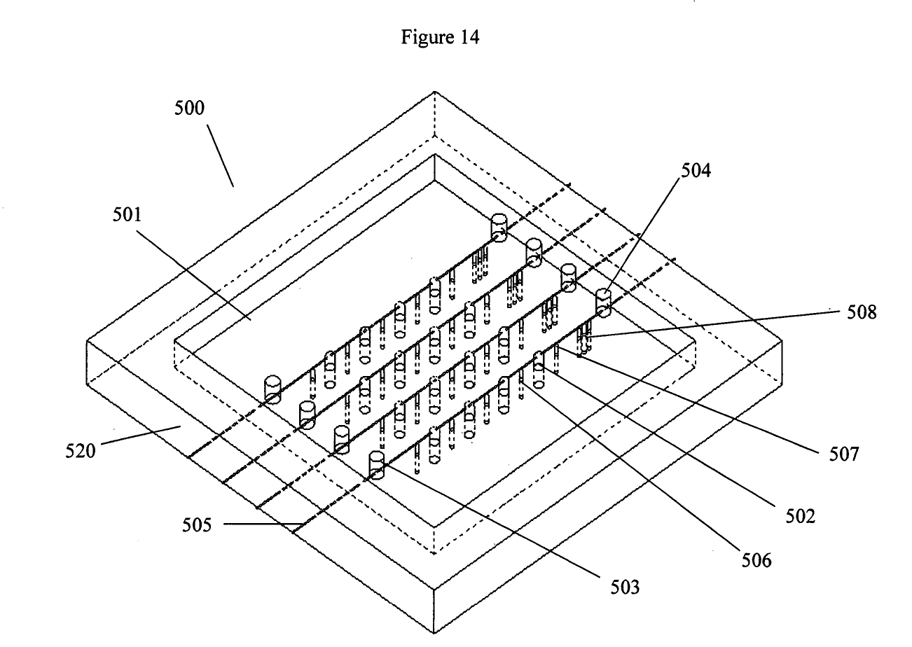

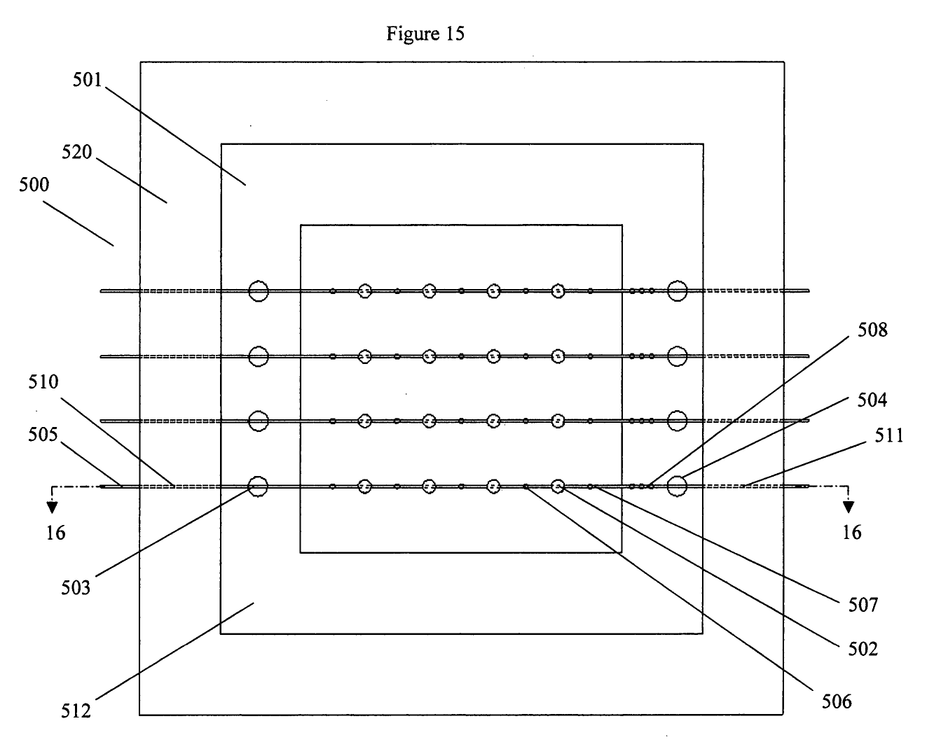

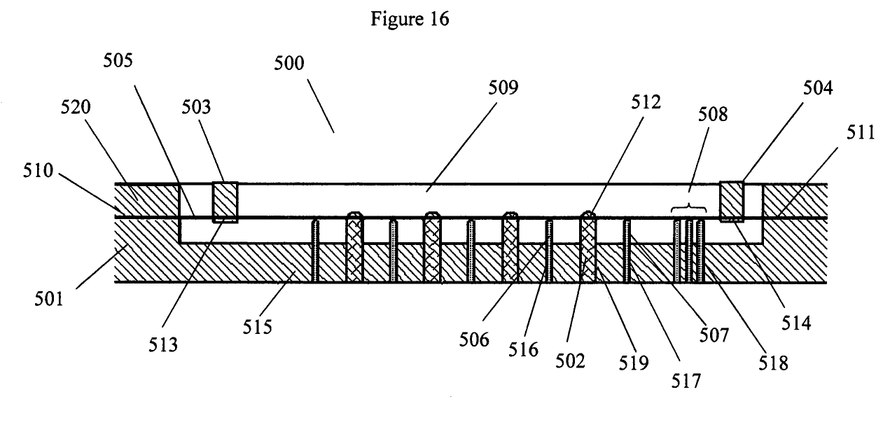

Referring to FIG. 14 to 16, methods with a device mold 500 is used to fabricate the array device 200. The methods with slight modifications on molds can be also used for the fabrication of the device 100 and 300. The device mold 500 is formed by assembling mold body 501 with mold components 502 to 508. The mold body 501 consists of sidewalls 520 and a base 515. The mold component 502 defines the recess 210 in the device body 201. The mold component 503 and 504 define the loading wells 203 and 204 in the device body 201. The elongated mold component 505 defines the channel 205 in the device body 201. The mold components 506 and 507 define the recesses 206 and 207 in the device body 201. On the sidewalls 520, there are slots 510 and 511 to accept the elongated mold component 505 to predefine the position of the channel 205 in the device 200. On the mold component 503 and 504 there is a slot 513 and 514. The elongated mold component 505 is passed through the slots 513 and 514 to position the mold components 503 and 504 in the device mold 500. On the dome of the mold component 502 there is also a slot 512. It is used to support the elongated mold component 505 at the predefined position and to create the connection of the channel 205 and the spot reservoir 209 in the device 200. The mold components 508 is used to create the recesses 216, 217, and 218 for the micropump 220 in the device 200. On the base 515 of the mold body 501, there are predefined holes 519 and 516-518 to accept the mold components 502 and 506-508. There is an open space 509 in the mold 500 for pouring liquid elastomeric material into it to cast the device 200 from the mold 500.

To create the deformable membranes 155 in the device 100 and 213 in the device 200, the set of mold components may include the mold component 506 and the elongated mold component 505. The two mold components may be placed in the device mold 500 with a gap between the tip of the mold component 506 and the elongated mold component 505. The gap can be designed from tens micrometers to hundreds micrometers according to the type of the linear motion actuators. For the piezoelectric motion component 150 and the thermopneumatic motion component 160, the displacement is relatively smaller. The gap should be in tens millimeters range, for example 30-90 micrometers. For the electric-mechanical motion actuator, the displacement is larger, the gap can be in hundreds micrometers range, for example 100-500 micrometers. For the slots 510 and 511 on the sidewall 520 and the slots 512 on the domes of the mold components 502, the elongated mold component 505 can be sustained straight and positioned. The gap can be kept consistent at the each position of the mold components 506 along the elongated mold component 505. After the device 100 or 200 is cast, the mold components are removed from the device mold and the device body, the recess 106 or 206 and the channel 105 or 205 are formed. So the deformable membrane is created.

To create the spot reservoir 109 with the two perpendicular channels in the device 100, a mold component with two perpendicular slots can be used. The two slots cross at a different level, one is positioned higher than another. When the device mold is assembled, two elongated mold components will be passed through the two holes to created perpendicular channels 105 and 111 at the spot reservoir 109.

To create the embedded spot reservoir 309 in the device 300, the set of mold components may include a castable and an elongated mold component. The castable mold component has a complementary shape suitable for forming the spot reservoir 309. The elongated mold component has a complementary shape suitable for forming the channel 305 connecting the spot reservoirs 309 in the device 300. On the castable mold component there is a slot with a same dimension and shape as the elongated mold component. The castable mold component may be made of a reversible, soluble, or sublimable material, such as fusible alloy, soap, wax, or ammonium salt.

To embed the substrate 302 inside the spot reservoir 309, the substrate 302 may be stuck on the bottom of the castable mold component. A post supporting the substrate 302 and the castable mold component is placed on the base of the device mold. The mold body may have a sidewall with a hole at a predefined position relative to the mold body. Assembling the device mold may include inserting the elongated mold component through the slot on the sidewall of the device mold, the slot on the castable mold component, and the slot on the opposite sidewall of the device mold. The elongated mold component aligns the castable mold component in the device mold. After the device body is released from the device mold and the elongated mold component and the post are pull out from the device mold, the channels 305 and 311 are created. A physical condition such as temperature may apply to the device body. The castable mold component inside the device body will be melted from a solid phase to a liquid or a gaseous phase, so that it can be removed through the channel 305 or 311 from the device body 301. Therefore the spot reservoir 309 is built inside the device body 301 and the substrate 302 is embedded.

Fabrication may include the following steps:

Step 1: Fabricating mold component and mold body

To create a recess such as the recess 206 in the device 200, the mold component 506 with a cylindrical tip can be machined from a wire. The mold component 502 with a dome tip can also be machined from a wire for creating the recess 210 in the device 200. The laser drilling can be used to create the slot 512 on the dome. The mold component 503 with the slot 513 can be also made from a wire and laser drilling. The elongated mold component can be made from a wire. The sidewall 520 and the base 515 can be made from conventional machining. The device mold can be open on the top for casting, like 500, or be a closed structure for injection casting.

If a castable mold component is used to create a embedded chamber such as the spot reservoir 309 in the device 300, the castable mold component may be fabricated from reversible, soluble, or sublimable material by a component mold. The reversible, sublimable, or soluble material is poured, injected, or compressed into the component mold. After the material solidifies, the castable mold component is separated from the component mold.

Step 2: Assembling the device mold

The device mold 500 is assembled by procedures below: placing the sidewall 520 on the base 515; inserting the mold components 502 and 506-508 into the holes 519 and 516-518 on the base 515; and passing the elongated mold component 505 through the slot 510 on the sidewall 520, the slot 513 on the mold component 503, the slots on the dome of the mold component 502, the slot 514 on the mold component 504, and the slot 511 on the sidewall 520.

Step 3: Coating the surfaces of the device mold

A mold release agent may be sprayed on the surface of the mold body 501 and the mold components assembled in it. This step is used to prevent adherence of the elastomeric material on the mold body 501 and the mold components when the mold body 501 and the mold components are removed from the device body 201. This step may be omitted if the adherence is not an issue.

Step 4: Casting the device body

Liquid elastomeric material is poured or injected into the device mold 500 to fill in the space defined by the mold body 501 and the mold components. The liquid elastomeric material is selected so that its curing temperature is lower than the melting (or sublimation) temperature of the castable mold components if the castable mold components are used.

Step 5: Removing the mold components from the device mold

First, the elongated mold component 505 is pull out from the device mold 500 through the slot 510 or 511. Then the mold components 503 and 504 can be removed from the top of the device mold 500. The mold components 502, and 506-508 can be pull out from the bottom of the device mold 500.

If the castable mold component is used, the device mold 500 with the device body 201 may be heated so that the castable mold component is melted (or vaporized), or a solvent may be used to inject to the soluble mold component to dissolve the mold components. The melted or dissolved mold component can be removed by use of vacuum suction or centrifuge.

Step 6: Releasing the device body from the mold body

The sidewall 520 can be removed from the base 515. The device body 201 can be then released from the mold base 515.

Step 7: Assembling the device 200 with functional components

The channel built by the elongated mold component 505 has an opening from the loading wells to the side of the device 200 after the device is released from the mold. Plugs will be inserted into the channels 205 to close this part of the channel. As an alternative method, liquid glue may be injected to this part of the channel instead.

Functional components, such as the glass substrate 102 and the pillars 208 will be assembled into the device body 101 or 201 at the predefined position. If the sealing between the device body and the functional components exhibits watertight, the device will be ready to use. Otherwise some glue may be applied to seal the functional components in the device body.

The techniques described herein the fluidic programmable array devices for microarray and microspot technology can be used in many different applications, including analytical chemistry, biological diagnosis, medical diagnosis, food testing, environment testing, biodefence, and drug detection and screening. Although some examples have been discussed above, other implementation and applications are also within the scope of the following claims.

1. A fluidic programmable array device, comprising:
an elastomeric body;
a substrate;
a recess;
a fluidic channel; and
two loading wells.

2. The device of the claim 1, wherein the recess is a cylinder with a dome.

3. The device of the claim 1, wherein a spot reservoir comprises the recess in the elastomeric body and a portion of the substrate at the recess.

4. The device of the claim 3, wherein the spot reservoir is connected with the fluidic channel.

5. The device of the claim 1, wherein the substrate is made of a material that is able to couple chemicals or molecules on it.

6. The device of the claim 1, wherein the surface of the substrate is coated a material that is able to couple chemicals or molecules on it.

7. The device of the claim 1, wherein the two loading wells are on both ends of the fluidic channel.

8. The device of the claim 1, further comprising more than the one recess to form more than one spot reservoirs with the substrate, and more than the one fluidic channel connecting the spot reservoirs, wherein the spot reservoirs and the fluidic channels form an array.

9. A structure in a device made of elastomeric material, comprising: a recess;
a fluidic channel;
a deformable membrane formed by the elastomeric material between a gap of the fluidic channel and the recess; and
the fluidic channel, the deformable membrane, and an actuator further form a pinch microvalve.

10. The device of the claim 9, wherein the recess has an elongated shape with a cylindrical top.

11. The device of the claim 9, wherein the actuator is a linear motion actuator inserted into the recess and touched the top of the recess.

12. The device of the claim 9, wherein the actuator embedded in a cavity formed by the recess and a substrate is a piezoelectric motion component with a head touched the top of the recess.

13. The device of the claim 9, wherein the actuator embedded in a cavity formed by the recess and a substrate is a magnetostrictive component with a head touched the top of the recess.

14. The device of the claim 9, wherein the actuator embedded in a cavity formed by the recess and a substrate is a thermopneumatic motion actuator, inside the cavity a liquid is filled and a microheater is built.

15. The device of the claim 9, wherein the at least three pinch microvalves form a peristaltic pump along the fluidic channel.

16. A fluidic programmable array device, comprising:
an elastomeric body;
a spot reservoir;
a fluidic channel; and
two loading wells.

17. The device of the claim 16, wherein the spot reservoir comprises a cavity formed by a recess in the elastomeric body and a pillar inlaid in the recess.

18. The device of the claim 17, wherein the top surface of the pillar is able to couple chemicals or molecules on it.

19. The device of the claim 17, wherein a through channel is in the center of the pillar to form a fluidic connecter to deliver a solution from outside of the device into the spot reservoir.

20. The device of the claim 16, wherein the spot reservoir is connected with the fluidic channel having the loading wells on the both ends.

21. The device of the claim 16, wherein the spot reservoir comprises a cavity inside the elastomeric body.

22. The device of the claim 21, wherein a channel in the elastomeric body connects the cavity from outside of the device.

23. The device of the claim 16, wherein the spot reservoir comprises a cavity inside the elastomeric body with an embedded substrate.

24. The device of the claim 23, wherein a center hole on the substrate and a channel in the elastomeric body connect the cavity from outside of the device.

25. The device of the claim 23, wherein a surface of the substrate is capable of coupling chemicals or molecules on it.

26. The device of the claim 16, wherein an array of the spot reservoirs is formed by more than the one spot reservoir and the one fluidic channel in the elastomeric body.
